SEQUENCE LISTING (12 sequences)

SEQ ID NO: 1 - DNA, 1446 bp, Escherichia coli
  CDS (1)..(1446): tldD
atgagtcttaacctggtaagtgaacaattgctagcggcgaacggcctgaaacatcaggacttgttcgcgatcctcggtcaactggccgaacgtcgccttgattatggcgatctctattttcagtcgagctatcacgaatcctgggttttagaagaccgcattattaaagatggttcttacaacatcgatcagggcgttggtgtgcgtgcaatcagcggtgaaaaaaccggatttgcttacgctgaccaaatcagcctgctggcgctggaacagagtgcgcaagcggcgcgcaccatcgtccgtgatagtggtgatggtaaagtacagacgctgggcgcggtagagcatagcccgttgtatacctcggtagatccgctgcaaagcatgagccgtgaagagaagctggatatcctgcgtcgcgtcgataaggttgcccgcgaagcggacaagcgcgtacaggaagtgactgccagcctcagtggtgtctatgaattaattttggttgcggccaccgacggcacgctagcggcggatgtccgtccgctggtgcgtctttccgtgagcgttctcgtcgaagaagatggcaaacgcgaacgcggtgccagtggcggcggcggtcgttttggttatgaattcttccttgccgatctcgacggcgaagtccgtgcggatgcatgggcaaaagaagcagtgcgtatggcgctggtcaatctttctgccgttgctgcaccagcgggcaccatgccggtagtacttggcgcaggttggccgggcgtgctgttgcatgaagcggttggtcacggtctggaaggcgacttcaaccgccgtggcacttcagtatttagtggacaggtcggggagctggtggcttcagaactgtgtaccgtggttgatgatggcacgatggtcgatcgccgaggttcggtggcgattgatgacgaaggtacgccaggccagtacaacgtgctgattgagaacggcattctgaaaggctacatgcaggataaactcaacgcgcgtttgatggggatgacgccgactggcaacggtcgccgtgaatcctacgcccatctgcccatgccgcgtatgaccaacacctatatgctgccgggtaaatcgaccccgcaggaaattattgaatccgttgagtacggtatctatgcaccgaactttggtggcggtcaggtggatatcacctccggcaaattcgttttctccacttcagaagcatatctgattgaaaacggtaaagtaacgaagccggtgaaaggcgcaacgttgattggttccggtatcgaaaccatgcagcagatttcgatggttggcaacgacctgaaactggataacggcgtgggtgtctgcggtaaagaagggcaaagtttgccggttggcgtgggccagccaacgttgaaagtcgataacctgactgttggcggtactgcgtaa

SEQ ID NO: 2 - protein, 481 aa, Escherichia coli
MSLNLVSEQLLAANGLKHQDLFAILGQLAERRLDYGDLYFQSSYHESWVLEDRIIKDGSYNIDQGVGVRAISGEKTGFAYADQISLLALEQSAQAARTIVRDSGDGKVQTLGAVEHSPLYTSVDPLQSMSREEKLDILRRVDKVAREADKRVQEVTASLSGVYELILVAATDGTLAADVRPLVRLSVSVLVEEDGKRERGASGGGGRFGYEFFLADLDGEVRADAWAKEAVRMALVNLSAVAAPAGTMPVVLGAGWPGVLLHEAVGHGLEGDFNRRGTSVFSGQVGELVASELCTVVDDGTMVDRRGSVAIDDEGTPGQYNVLIENGILKGYMQDKLNARLMGMTPTGNGRRESYAHLPMPRMTNTYMLPGKSTPQEIIESVEYGIYAPNFGGGQVDITSGKFVFSTSEAYLIENGKVTKPVKGATLIGSGIETMQQISMVGNDLKLDNGVGVCGKEGQSLPVGVGQPTLKVDNLTVGGTA

SEQ ID NO: 3 - DNA, 1353 bp, Escherichia coli
  CDS (1)..(1353): tlDE
atggcacttgcaatgaaagtaatctctcaagttgaagcgcagcgcaagattctggaagaagcagtttccactgcgctggagttggcctcaggcaaatcggacggtgcggaagttgccgtcagcaagaccaccggcattagcgtaagcacgcgttatggtgaagtggagaatgttgaattcaatagcgatggcgcgctggggatcactgtttatcaccagaaccgcaaaggtagcgcatcatccaccgatttaagcccgcaggccattgcccgtactgtacaggcggcgctggatattgcccgttatacctcgccagatccctgtgccggcgtggcagacaaagagctgctggcctttgacgcaccagatctcgacttgttccaccctgcggaagtttccccggatgaagccattgaactggcggcccgcgcagaacaggcggcattgcaggcggacaaacgcatcaccaataccgaaggtggcagctttaacagccactacggtgtcaaagtttttggcaacagccacggcatgttgcagggttactgctcaacgcgtcattcgctctccagctgtgtaattgccgaagaaaatggcgatatggagcgtgattacgcctacaccattggtcgtgcgatgagcgatctgcaaacgccagagtgggttggggccgactgtgctcgccgtactttatcgcgtctgtcaccgcgtaaactctccaccatgaaagcgccagtcatttttgccaatgaagtggcaaccgggctttttgggcatctggtgggggcgatagcgggtggatcggtttatcgtaaatctaccttcctgctggattcgctgggtaaacaaattctgccggactggctgaccattgaagagcatccgcatctgctgaaagggctggcgtcgacgccattcgacagcgaaggtgtgcgcaccgagcgtcgcgatattattaaagatggcatcctgactcagtggctgctgaccagctactcggcgcggaaactggggctgaaaagcaccggacatgcgggcggtattcacaactggcggattgccggacaaggtctaagcttcgagcagatgctcaaagagatgggcaccgggctggtggtgacggaattgatgggccagggcgtgagtgccattaccggtgattattcccgtggtgcagcgggcttctgggtagagaatggcgaaattcagtatccggtgagcgaaatcaccatcgcaggtaatttaaaagatatgtggcgcaatattgtcaccgtcggtaacgatattgaaacacgcagtaatatacagtgtggttctgtgctgttgccggagatgaaaatcgccggacagtaa

SEQ ID NO: 4 - protein, 450 aa, Escherichia coli
MALAMKVISQVEAQRKILEEAVSTALELASGKSDGAEVAVSKTTGISVSTRYGEVENVEFNSDGALGITVYHQNRKGSASSTDLSPQAIARTVQAALDIARYTSPDPCAGVADKELLAFDAPDLDLFHPAEVSPDEAIELAARAEQAALQADKRITNTEGGSFNSHYGVKVFGNSHGMLQGYCSTRHSLSSCVIAEENGDMERDYAYTIGRAMSDLQTPEWVGADCARRTLSRLSPRKLSTMKAPVIFANEVATGLFGHLVGAIAGGSVYRKSTFLLDSLGKQILPDWLTIEEHPHLLKGLASTPFDSEGVRTERRDIIKDGILTQWLLTSYSARKLGLKSTGHAGGIHNWRIAGQGLSFEQMLKEMGTGLVVTELMGQGVSAITGDYSRGAAGFWVENGEIQYPVSEITIAGNLKDMWRNIVTVGNDIETRSNIQCGSVLLPEMKIAGQ

SEQ ID NO: 5 - DNA, 837 bp, Escherichia coli
  CDS (1)..(837): dam
atgaagaaaaatcgcgcttttttgaagtgggcagggggcaagtatcccctgcttgatgatattaaacggcatttgcccaagggcgaatgtctggttgagccttttgtaggtgccgggtcggtgtttctcaacaccgacttttctcgttatatccttgccgatatcaatagcgacctgatcagtctctataacattgtgaagatgcgtactgatgagtacgtacaggccgcacgcgagctgtttgttcccgaaacaaattgcgccgaggtttactatcagttccgcgaagagttcaacaaaagccaggatccgttccgtcgggcggtactgtttttatatttgaaccgctacggttacaacggcctgtgtcgttacaatctgcgcggtgagtttaacgtgccgttcggccgctacaaaaaaccctatttcccggaagcagagttgtatcacttcgctgaaaaagcgcagaatgcctttttctattgtgagtcttacgccgatagcatggcgcgcgcagatgatgcatccgtcgtctattgcgatccgccttatgcaccgctgtctgcgaccgccaactttacggcgtatcacacaaacagttttacgcttgaacaacaagcgcatctggcggagatcgccgaaggtctggttgagcgccatattccagtgctgatctccaatcacgatacgatgttaacgcgtgagtggtatcagcgcgcaaaattgcatgtcgtcaaagttcgacgcagtataagcagcaacggcggcacacgtaaaaaggtggacgaactgctggctttgtacaaaccaggagtcgtttcacccgcgaaaaaataa

SEQ ID NO: 6 - protein, 278 aa, Escherichia coli
MKKNRAFLKWAGGKYPLLDDIKRHLPKGECLVEPFVGAGSVFLNTDFSRYILADINSDLISLYNIVKMRTDEYVQAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDASVVYCDPPYAPLSATANFTAYHTNSFTLEQQAHLAEIAEGLVERHIPVLISNHDTMLTREWYQRAKLHVVKVRRSISSNGGTRKKVDELLALYKPGVVSPAKK

SEQ ID NO: 7 - DNA, 178 bp, Escherichia coli
  CDS (1)..(177): Truncated dam
atgaagaaaaatcgcgcttttttgaagtgggcagggggcaagtatcccctgcttgatgatattaaacggcatttgcccaagggcgaatgtctggttgagccttttgtaggtgccgggtcggtgtttctcaacaccgacttttctcgttatatccttgccgatatcaatagcgacctga

SEQ ID NO: 8 - protein, 59 aa, Escherichia coli
MKKNRAFLKWAGGKYPLLDDIKRHLPKGECLVEPFVGAGSVFLNTDFSRYILADINSDL

SEQ ID NO: 9 - DNA, 186 bp, Escherichia coli
  CDS (1)..(186): csrA
atgctgattctgactcgtcgagttggtgagaccctcatgattggggatgaggtcaccgtgacagttttaggggtaaagggcaaccaggtacgtattggcgtaaatgccccgaaggaagtttctgttcaccgtgaagagatctaccagcgtatccaggctgaaaaatcccagcagtccagttactaa

SEQ ID NO: 10 - protein, 61 aa, Escherichia coli
MLILTRRVGETLMIGDEVTVTVLGVKGNQVRIGVNAPKEVSVHREEIYQRIQAEKSQQSSY

SEQ ID NO: 11 - DNA, 153 bp, Escherichia coli
  CDS (1)..(153): csrA 1-50
atgctgattctgactcgtcgagttggtgagaccctcatgattggggatgaggtcaccgtgacagttttaggggtaaagggcaaccaggtacgtattggcgtaaatgccccgaaggaagtttctgttcaccgtgaagagatctaccagcgttaa

SEQ ID NO: 12 - protein, 50 aa, Escherichia coli
MLILTRRVGETLMIGDEVTVTVLGVKGNQVRIGVNAPKEVSVHREEIYQR

(19) United States
(12) Patent Application Publication
(10) Pub. No.: US 2013/0109061 A1
(43) Pub. Date: May 2, 2013

(54) HYPERPROLIFERATIVE RECOMBINANT CELL

(71) Applicants: Laurence Van Melderen, Waterloo (BE); Johan Timmermans, Waterloo (BE)

(72) Inventors: Laurence Van Melderen, Waterloo (BE); Johan Timmermans, Waterloo (BE)

(73) Assignee: UNIVERSITE LIBRE DE BRUXELLES, Bruxelles (BE)

(21) Appl. No.: 13/699,273

(22) PCT Filed: May 20, 2011
(86) PCT No.: PCT/EP11/58260

§ 371 Date: Jan. 2, 2013

(30) Foreign Application Priority Data
May 20, 2010 (EP) 10163490.5

(51) Int. Cl. C12N 15/74

(52) U.S. Cl. CPC C12N 15/74
USPC 435/91.4; 435/471; 435/252.3; 435/101

(57) ABSTRACT

A method increases the growth of a cell by a deletion or one or more mutation(s) in the tldD and/or tldE genes, coupled with one or more mutation(s) in dam or a partial deletion or deletion of the dam gene. A cell is obtained by the method and used.

### FIELD OF THE INVENTION

The present invention is related to a hyper-proliferative (recombinant) cell, its preparation process and its use, especially for the production of molecules of interest.

Background of the invention and state of the art

Recombinant micro-organisms are used as such (in the form of a fertilizer, of a probiotic, of a vaccine, etc) or for the production of a variety of molecules, such as DNA constructs, polypeptides, etc in a cheap and flexible way.

However, a better yield of production requires culture of the micro-organisms, especially bacterial cells, at high densities and remains a difficulty.

Furthermore, fermentation is a consequence of micro-organism culture in broth, and results into large production of acetate, especially when the glycolysis overflows the Krebs cycle, inhibiting in turn their growth.

Several possibilities were implemented, such as dialysis, filtration and pure oxygen addition. The acetate separation from a micro-organism by dialysis or filtration allows the latter a further growth, with limitations, such as the price and a possible dilution of the obtained products in the medium. Oxygen addition favours oxidative metabolism instead of glycolysis, as well as acetate consumption. However, this method requires more sophisticated devices to be added to bioreactors.

The European Patent application EP0316229 describes a mutated Escherichia coli strain presenting reduced acetate production and increased cell density and subsequent yield. However, in this patent application, the obtained mutation(s), and the affected pathway(s) are not disclosed.

TldD and tldE genes were identified as necessary for microcin B17 processing and maturation (Allali, N., Afif, H., Couturier, M. and Van Melderen, L. (2002) The highly conserved TldD and TldE proteins of Escherichia coli are involved in microcin B17 processing and in CcdA degradation. J. Bacteriol, 184, 3224-3231). As the TldD and TldE proteins are highly conserved in many eu- and archae-bacteria, this suggests that they have other central roles.

The methylase encoded by the dam gene (Dam methylase) transfers a methyl group from S-adenosylmethionine to the N6 position of the adenine residues in the sequence GATC.

Dam is involved in the resistance towards specific endonucleases, in the mechanism of repair of DNA and in the control of gene expression.

### SUMMARY OF THE INVENTION

The present invention provides new recombinant cells and their preparation process that do not present the drawbacks of the state of the art.

Preferably, the present invention provides such cells that present improved properties, like a reduced acetate production and a reduced sensitivity to acetate, cells presenting an extended logarithmic growth phase and giving rise to a higher number of viable cells compared to the wild-type strain for improving (increasing) the production of valuable biological or chemical downstream products which mean endogenous and exogenous molecules obtained from these cells, like nucleic acid constructs, polypeptides, saccharides, lipids, vitamins, etc.

Furthermore, the present invention provides cells having increased plasmid amount.

A first aspect of the present invention is related to a method for improving (increasing) the growth of a (prokaryote) cell, wherein this cell (wild-type cell) that (natively) comprises the (wild-type and/or functional) tldD and/or tldE, and dam nucleotide sequences is submitted to a partial or total deletion of the said tldD and/or tldE, and dam nucleotide sequence(s) or to one or more mutation(s) inactivating (the function of) TldD and/or TldE, and Dam proteins.

These mutation (s) include mutation (s) in these tldD and/or tldE, and dam nucleotide sequence(s), preferably causing insertion or deletion of a nucleotide, and/or introducing a stop codon and/or resulting in a translated peptide having a substitution of one amino-acid by another, but also (possibly) mutation(s) of nucleotide sequence(s) acting downstream and/or upstream these tldD and/or tldE, and dam sequences and resulting in the same phenotype.

Preferably, the mutation inactivating the Dam protein is partially inactivating its activity.

Partial inactivation preferably means truncation of the protein retaining the N-terminal part of the wild-type protein and possibly giving rise to a less active protein as compared to the wild-type activity.

According to the invention, one mutation inactivating the Dam protein is (preferably) the dam::kan mutation.

Advantageously, the deletion of the 3′ region of the dam sequence gives rise to a sequence composed of the 5′ region of dam native sequence, more preferably a sequence starting from nucleotide 1 up to nucleotide 178 and encoding a polypeptide called Dam 1-59 being composed of the 59 amino-terminal amino acids of the native Dam protein (of E. coli; SEQ. ID. NO. 8).

This nucleotide sequence is called the short dam sequence.

The short dam sequence (of E. coli; SEQ. ID. NO. 7) lacks the 3′ end of the dam native sequence from nucleotide 179 to nucleotide 837. The same mutation is obtained in the dam::kan mutant.

Alternatively, mutation (s) in the dam sequence can be the full deletion or mutation(s) fully inactivating its activity.

Advantageously, the present method for improving (increasing) the growth of a (prokaryote) cell further comprises the step of submitting the (prokaryote) cell to the deletion of the 3′ part of the csrA nucleotide sequence or to one or more mutation(s) reducing the activity of (partially inactivating) this CsrA protein.

Preferably, the deletion of the 3′ region of the csrA sequence gives rise to a sequence composed of the 5′ region of csrA native sequence, more preferably a sequence starting from nucleotide 1 up to nucleotide 150 and encoding a polypeptide called CsrA 1-50 being composed of the 50 amino-terminal amino acids of the native CsrA protein.

This nucleotide sequence is called the short csrA sequence.

The short csrA sequence lacks the 3′ end of the csrA native sequence from nucleotide 151 to nucleotide 186.

Preferably, in this method for improving (increasing) the growth of a (prokaryote) cell, this (prokaryote) cell is grown in a medium supplemented with a carbon source, being preferably a glycolytic carbon source and/or selected from the group consisting of glycerol, pyruvate and/or a glycolytic saccharide, more preferably glucose.

A related aspect of the present invention concerns a method for increasing the plasmid amount (relative abundance; μg plasmid DNA:μg chromosomal DNA) in a (prokaryote) cell comprising the step of submitting tldD and/or tldE gene(s) (of this (prokaryote) cell) to one or more mutation(s) inactivating TldD and/or TldE proteins.

Preferably, in this method for increasing the plasmid amount, the plasmid amount of the (recombinant) cell submitted to one or more mutation(s) inactivating TldD and/or TldE proteins is increased by at least 3-fold, preferably by at least 10-fold compared to a control cell (reference or wild type cell).

These mutation(s) include mutation(s) in the tldD and/or tldE nucleotide sequence(s), preferably causing insertion or deletion of a nucleotide, and/or introducing a stop codon and/or resulting in a translated peptide having a substitution of one amino-acid by another, but also mutation(s) of nucleotide sequence(s) acting downstream and/or upstream these tldD and/or tldE sequences and resulting in the same phenotype.

The preferred mutations inactivating TldD and/or TldE proteins is (are) the full deletion of these tldD and/or tldE gene(s).

Preferably, in this method for increasing the plasmid amount, the plasmid amount is increased by at least 3-fold.

Advantageously, in this method for increasing the plasmid amount, the plasmid is a low copy-number plasmid (i.e. a plasmid that is present under normal condition at less than 50 copies per cells, preferably, less than 20 copies per cell, more preferably, less than 15, 10, 9, 8, 7, 6, or even less than 5 copies per cell).

Preferably, this method for increasing the plasmid amount further comprises the step of submitting the (prokaryote) cell to a partial or to a total deletion of dam and/or of csrA nucleotide sequence or to one or more mutation(s) inactivating the activity of the produced Dam and/or CsrA proteins.

Preferably, these mutations inactivating Dam and/or CsrA protein are the deletion of the 3′ part of the dam nucleotide sequence and/or the deletion of the 3′ part of the csrA nucleotide sequence, resulting into the production of truncated (short) Dam and/or of truncated (short) CsrA proteins.

Preferably, in this method for increasing the plasmid amount, the (prokaryote) cell is grown in a medium supplemented with a carbon source, being preferably selected from the group consisting of a glycolytic carbon source and/or selected from the group consisting of glycerol, pyruvate and/or a glycolytic saccharide, more preferably glucose.

Another aspect of the invention concerns an isolated (purified) hyperproliferative (recombinant) (prokaryote) cell wherein the tldD and/or tldE, and dam nucleotide sequence(s) are partially or totally deleted or comprise one or more mutation(s) inactivating this tldD and/or tldE, and dam nucleotide sequences (preferably wherein this mutation inactivating Dam, such as the deletion of the 3′ part of the dam nucleotide sequence, is the short dam sequence comprising the 5′ region of the dam open reading frame starting from nucleotide 1 up to nucleotide 178 or one or more mutations inactivating the Dam protein, preferably the dam::kan mutation) and wherein the cell possibly further comprises an exogenous nucleotide sequence encoding a gene product of interest.

Possibly, this prokaryote cell is not E. coli.

These mutation(s) include mutation(s) in these tldD and/or tldE, and dam nucleotide sequence(s) for avoiding their expression, but also mutation (s) of nucleotide sequence(s) acting downstream and/or upstream these tldD and/or tldE, and dam sequences and resulting in the same phenotype.

Preferably, this cell is selected from the group consisting of Clostridium sp., Sphingomonas sp., Bacillus sp. and possibly the Lactobacillus sp., Bifidobacterium sp., Lactococcus sp. cells.

A further aspect of the present invention is related to an isolated (and purified) hyperproliferative (recombinant) E. coli cell, wherein the tldD and/or tldE, and dam sequences (SEQ. ID. NO. 1, SEQ. ID. NO. 3, SEQ. ID. NO. 5) are deleted or comprise one or more mutations inactivating the TldD and/or TldE, and Dam activities, (preferably wherein this mutation inactivating Dam, such as the deletion of the 3′ part of the dam nucleotide sequence, is the short dam sequence comprising the 5′ region of the dam open reading frame starting from nucleotide 1 up to nucleotide 178 (SEQ. ID. NO. 7) or one or more mutations inactivating the Dam protein (and/or the dam::kan mutation)) and wherein the cell further comprises an exogenous insert or an exogenous nucleotide sequence encoding a gene product of interest.

These mutation(s) include mutation(s) in the tldD and/or tldE, and dam nucleotide sequence(s) for reducing or avoiding their expression, but also mutation(s) of nucleotide sequence(s) acting downstream and/or upstream these tldD and/or tldE, and dam sequences and resulting in the same phenotype.

“A exogenous nucleotide sequence encoding for a gene product of interest or insert” refers to nucleotide sequences possibly involved in the production of biological or chemical compounds being these gene products of interest, that are not natively present in the (wild-type) cell and which presents an interest for industrial, medical, cosmetic, chemical or environmental applications. Non-limiting examples of these genes products of interest are nucleotides sequences (possibly comprised in vectors, such as plasmids, viruses or phagemids), saccharides (including oligo and polysaccharides), lipids (including fatty acids, such as omega-3 fatty acids), biopolymers, acids (including butyric acid), vitamins, hydrocarbons and derived products including polymers, hemes, enzymes and co-enzymes, bacteriocins, antibodies (or portions thereof, including nanobodies) nucleotide-based vaccination composition, (poly)peptides (or their portions, such as epitopes) for vaccination and any protein that may be recovered for any industrial, medical, cosmetic, chemical, food-related or environmental application.

Advantageously, the gene product of interest is an immunologically active gene product (immuno-suppressive compound or immuno-stimulative compound) or may consist in a compound that is effective in degradation of an environmental pollutant, preferably a pesticidally active product.

The gene product of interest may also comprise or consist of polypeptides, such as antigens or portions of antigens, enzymes, anti-thrombolytic compounds, hormones, neurotransmitters, antibodies, portion of antibodies, nanobodies, proteins involved in the synthesis of saccharides, vitamins, lipids, proteins involved in the transport of heavy metals, adjuvants to vaccine, etc.

Advantageously, these hyperproliferative (prokaryote) cells (encoding wild-type and/or functional CsrA protein; SEQ. ID. NO. 9 for E. coli) have further been submitted to the deletion of the 3′ part of the csrA nucleotide sequence or to one or more mutation(s) reducing the activity (partially inactivating) of this CsrA protein.

Preferably, the deletion of the 3′ region of the csrA sequence gives rise to a sequence composed of the 5′ region of csrA native sequence, more preferably a sequence starting from nucleotide 1 up to nucleotide 150 and encoding a polypeptide called CsrA 1-50 being composed of the 50 amino-terminal amino acids of the native CsrA protein (SEQ. ID. NO. 11 for E. coli).

This nucleotide sequence is called the short csrA sequence.

The short csrA sequence lacks the 3′ end of the csrA native sequence from nucleotide 151 to nucleotide 186. The same mutation is obtained in the csrA::kan mutant

Furthermore, this recombinant (prokaryote) cell may also further comprise one or more selection markers.

“A selection marker” refers to a gene that confers an advantage to a cell under selective pressure.

The inventors have also observed that the glycogen content of the recombinant cell of the invention is increased, by 10 fold, preferably by at least 50 fold compared to a control (wild type) cell which does not comprise these genetic modifications.

Furthermore, the inventors have observed that the recombinant (prokaryote) cell of the invention presents also other phenotypes, such as a reduced acetate production. Preferably, in the recombinant (prokaryote) cell of the invention, the acetate production is reduced by a factor of at least 10%, preferably of at least 15%, more preferably of at least 20%, compared to a production of a control (wild type) cell (a cell which does not present these genetic modifications).

The inventors have observed that the hyperproliferative cell of the invention presents an extended exponential growth, which means that its growth is increased by at least 25% or 50%, preferably by at least 75%, more preferably by 100% compared to the growth of a control (wild type) cell that does not comprise these genetic modifications.

Advantageously, the lag period and the generation time remain unaffected in the hyperproliferative cell according to the invention.

Surprisingly, the sensitivity of the hyperproliferative cell of the invention towards acetate is also reduced, preferably by a factor of at least 30%, more preferably of at least 50%, and advantageously of at least 75% compared to a control (wild type) cell that does not comprise these genetic modifications.

Therefore, another aspect of the present invention concerns method for obtaining a reduction of acetate production by a (recombinant) cell, preferably a reduction by a factor of at least 10%, preferably at least 15%, more preferably at least 20%, and/or a method that reduces the sensitivity of this cell towards acetate by a factor of at least 30%, more preferably at least 50%, advantageously by at least 75%, compared to a control (reference or wild type) cell which comprises natively the tldD and/or tldE, and dam nucleotide sequences and wherein this cell is submitted to a partial or total deletion of this tldD and/or tldE, and of dam sequence(s) or to one or more mutation(s) inactivating these nucleotide sequences (i.e inactivating the corresponding encoded protein).

The present invention is also related to a method to increase the glycogen content of a (recombinant) cell (increased by 10 fold, preferably by at least 50 fold) compared to a control (reference or wild type) cell which comprises natively the tldD and/or tldE, and dam nucleotide sequences and wherein this cell is submitted to a partial or total deletion of this tldD and/or tldE, and of dam sequence(s) or to one or more mutation(s) inactivating these nucleotide sequence (i.e. inactivating the corresponding encoded protein, such as the deletion of the 3′ part of the dam nucleotide sequence or to one or more mutation(s) reducing the activity of the Dam protein (partially inactivating the Dam protein activity).

Conversely, the present invention is related to the use of the recombinant cell (of the present invention) for the (increased) production of glycogen.

These mutation(s) include mutation(s) in the tldD and/or tldE, and of dam nucleotide sequences for avoiding their expression, but also mutation(s) of nucleotide sequence(s) acting downstream and/or upstream these tldD and/or tldE and dam sequences and resulting in the same phenotype.

Preferably, the mutation inactivating the dam sequence is the dam::kan mutation and/or the deletion of the 3′ part of the dam nucleotide results in the dam 1-178 sequence comprising the 5′ region of the dam open reading frame starting from nucleotide 1 up to nucleotide 178.

In the method(s) according to the invention, the (prokaryote) cell is (are) preferably grown in a medium supplemented with a carbon source, preferably a source selected from the group consisting of glycolytic carbon source and/or selected from the group consisting of glycerol, pyruvate and/or a glycolytic saccharide (such as glucose, fructose, sucrose, arabinose, galactose or lactose, more preferably glucose), more preferably the medium is supplemented with glucose.

Another aspect of the invention is the use of the hyperproliferative cell according to the invention for synthesis of a molecule of interest, being preferably selected from the group consisting of RNA or DNA sequences, polypeptides (preferably insulin), especially antigens or portions of antigens, enzymes (including xyloglucanases, xylanases, lipases, esterases, etc), hormones, vitamins, hemes, lipids, amino-acids, acids (such as butyric acid), saccharides (oligo and/or polysaccharides), biopolymers, hydrocarbons, carbohydrates (such as glycogen), antibiotic molecules, plasmids, viruses, phagemids or a mixture thereof.

The hyperproliferative cell according to the invention may also be used as a probiotic or a bioremediator and a last aspect of the invention is related to a pharmaceutical, cosmetical, neutraceutical, food, feed or beverage composition or a waste treatment plant comprising the cell according to the invention.

The hyper-proliferative cell according to the invention (presenting an important glycogen storage) can be used for the synthesis of biological (biodegradable) plastic, for the production of biological (biodegradable) plastic bags or other devices made by ‘plastic’, possibly by using the glycogen as starting material for the synthesis of this biological (biodegradable) plastic but also in the manufacture of paper (papermaking, paper coating with improved properties (smoothness, whiteness, etc), . . . ), food products (food additives used as thickeners and stabilizers), adhesives and glues (preferably corrugated board adhesives), in gypsum wall board manufacturing process, in textile chemicals (to reduce breaking of yarns during weaving or as textile printing thickener), in printing industry (for the manufacture of anti-set-off spray powder) and in oil exploitation (to adjust viscosity of drilling fluid).

A related aspect of the present invention concerns the use of a (prokaryote) cell (such as E. coli) having been submitted to one or more mutation(s) in tldD and/or tldE nucleotide sequence(s) encoding TldD and/or TldE proteins, these mutations inactivating these TldD and/or TldE proteins for increasing the plasmid amount (relative abundance; i.e. μg plasmid DNA:μg chromosomal DNA).

These mutation (s) include mutation (s) in these tldD and/or tldE nucleotide sequence(s), preferably causing insertion or deletion of a nucleotide, and/or introducing a stop codon and/or resulting in a translated peptide having a substitution of one amino-acid by another, but also mutation(s) of nucleotide sequence(s) acting downstream and/or upstream these tldD and/or tldE sequences and resulting in the same phenotype.

The preferred mutations inactivating TldD and/or TldE proteins is (are) the full deletion of these tldD and/or tldE gene(s).

Preferably, in these mutated (prokaryote) cells, the plasmid amount is increased by at least 3 times.

Advantageously, this plasmid is a low copy plasmid (i.e. a plasmid that is present under normal condition at less than 50 copies per cells, preferably, less than 20 copies per cell, more preferably, less than 15, 10, 9, 8, 7, 6, or even less than 5 copies per cell.

Possibly, in this use for increasing the plasmid amount, the (prokaryote) cell has further been submitted to one or more mutation (partially) inactivating Dam protein and/or to one or more mutation (partially) inactivating CsrA protein.

Preferably, these mutations inactivating Dam and/or CsrA protein are the deletion of the 3′ part of the dam nucleotide sequence and/or the deletion of the 3′ part of the csrA nucleotide sequence, resulting into the production of truncated Dam and/or of CsrA proteins.

### DETAILED DESCRIPTION OF THE INVENTION

### Example 1

### Establishment and Characterization of the Strain According to the Invention

Escherichia coli cells were engineered to have the deletion of tldD gene (SEQ. ID. NO 1) and/or tldE gene (SEQ. ID. NO 3) and further with the replacement of dam sequence (SEQ. ID. NO 5) by the dam 1-178 nucleotide sequence, being deleted of the 3′ part of the dam native sequence and encoding the Dam polypeptide 1-59 (SEQ. ID. NO 7 and 8). The sequence of this short dam 1-178 is:

5′ ATGAAGAAAAATCGCGCTTTTTTGAAGTGGGCAGGGGGCAAGTATCCCCTGCTTGAT
GATATTAAACGGCATTTGCCCAAGGGCGAATGTCTGGTTGAGCCTTTTGTAGGTGCCGG
GTCGGTGTTTCTCAACACCGACTTTTCTCGTTATATCCTTGCCGATATCAATAGCGACC
TGA 3′

Similar modification can be engineered in other prokaryote cells having corresponding nucleotide sequences.

MG1655 (wild-type, laboratory K-12 strain) cells were engineered by removing tldD and tldE Open Reading Frames (ORFs) (delta tldD mutant and delta tldE mutant, respectively) from the start codon up to the last codon (leaving the stop codon) using the Datsenko and Wanner method (Datsenko, K. A. and Wanner, B. L. (2000) One-step inactivation of chromosomal genes in Escherichia coli K-12 using PCR products. Proc. Natl. Acad. Sci. USA. June 6; 97(12):6640-5).

These three obtained single mutants have the same growth properties in LB and in minimal medium supplemented with glucose or other carbon sources than the corresponding wild-type cell strain.

The combination of the short (truncated) dam sequence and either delta tldD or delta tldE mutants, hereafter referred to as “double mutants”, results into a growth comparable to the wild type strain in LB medium but, very surprisingly, into a two-fold increase growth in glucose-enriched medium.

The characteristic of the mutant presenting the deletion of the 3′ part of the dam nucleotide sequence and presenting a sequence encoding this short (truncated) Dam polypeptide and both tldD and tldE sequences (genes) deletion, as hereafter referred to as “triple mutant”.

The addition of other carbon sources passing through glycolysis (i.e. glycolytic carbon sources), such as arabinose (via fructose-6-phosphate) and/or other carbon sources such as glycerol and pyruvate provoke an increased growth, but to a lesser extend than glycolytic saccharides such as glucose.

In addition, glycogen storage of the mutant strain is advantageously increased by about 50-fold compared to a reference (control or wild type cell above described).

The synthesis of glycogen could be also used as a substrate for the productions of derived saccharides, such as maltodextrins that find some advantageous applications in the food industry.

Very surprisingly is the fact that, for the same glucose consumption, these double and triple (and further) mutants of the invention produce less acetate, i.e. 0.6 g/l versus 0.9 g/l for wild-type strain (control or reference).

Acetate production is measured using K-Acet kit (Megazyme). The skilled one may easily find other methods and adapt them to the present invention.

Also very surprising is the increased resistance (to 0.9 g/l) to acetate-inhibition of growth by the double and triple (and further) mutants in comparison to the wild-type strain (control or reference) (growth inhibited at 0.5 g/l).

Preferably, the hyper-proliferative cells (bacteria) of the present invention may further comprise selection markers.

### Example 2

### Use of the Modified Cell Strains According to the Invention

According to the invention, recombinant cells (bacteria including Escherichia coli strains) were further developed incorporating an exogeneous (exogenous sequence or insert not present natively in the cell) nucleotide sequence for expressing a protein of interest and advantageously the inventors measured the increased production of the corresponding protein.

The inventors have used recombinant bacteria according to the invention, preferably Escherichia coli strains, for the production of polypeptides, preferably insulin, and enzymes, including xyloglucanase, lipase and esterases.

The inventors have used recombinant bacteria according to the invention, preferably Escherichia coli strains, for the production of amino-acids, such as phenylalanine.

The inventors have used modified bacteria according to the invention for the production of peptidic and non-peptidic hormones.

Furthermore, the inventors have used recombinant bacteria according to the invention for the production of vitamins and coenzymes, such as menaquinones (vitamin K2), vitamin B12, coenzyme Q₁₀ and heme group.

The inventors have used modified bacteria according to the invention for the production of lipids, especially those comprising omega-3 fatty acids.

The inventors have used recombinant bacteria, such as Clostridium strains, as described in WO2007/095215 for the production of acids, such as butyric acid. Surprisingly, the inventors measured that the reduction of acetate synthesis of this recombinant bacteria, coupled to an increased growth, synergize to increase the yield of butyric acid. Advantageously, the inventors noticed that butyric acid may be converted into hexane after electrolysis.

The inventors have used recombinant bacteria according to the invention for the production of oligo and/or polysaccharides, such as xanthan gum, by Xanthomonas campestris and/or of other biopolymers.

The inventors have obtained recombinant Sphingomonas strains for the increased production of sphingan and/or of Rhizobium meliloti strains for the synthesis of succinoglycan polysaccharides.

The inventors have obtained recombinant Escherichia coli strains with nucleotide sequences described in WO2008/021141 for the increased production of ethanol from lignocellulosic biomass.

The inventors have obtained Escherichia coli strains according to the invention and further transformed with nucleotide sequences described in WO2008/003078 for the increased production of isoprenoid lipids. They adapt these methods to other prokaryotes, including photosynthetic species.

The inventors have modified Escherichia coli strains according to the invention and further with nucleotide sequences as described in Klocke et al. (Applied Genetics and Molecular Biotechnology, 67, 532-538 (2005)) for the production of bacteriocins (enterocin A and B).

The inventors have modified Escherichia coli strains according to the invention for an increased production of plasmids or fragments thereof that may comprise recombinant nucleotide sequences.

The person skilled in the art may also modify bacteria strains that are used as probiotics (live micro-organisms which, when administrated in adequate amounts, confer a health benefit on the host, especially in mammals including humans) or bio-remediators.

The person skilled in the art may use the modified bacteria according to the invention for an increased production of adjuvants to vaccines by further isolating DNA fragment sequences bearing immunostimulatory properties (such as CpG rich sequences) and possibly saccharides derivatives.

Besides an increased growth useful in the industrial processes, the inventors noticed an increased growth of the probiotic micro-organism in vivo.

### Example 3

### Comparison of the Modified Cell Strains According to the Invention with Other Hyperproliferative Cell and Development of Another Hyperproliferative Cell

The inventors have further performed the comparison of the hyperproliferative cell of the present invention having been submitted to a tldD and tldE deletion and to a Dam truncation with another hyperproliferative cell they have previously developed, having been submitted to a tldD and tldE deletion and to a CsrA truncation. They have further combined all these tldD, tldE, Dam, CsrA mutations.

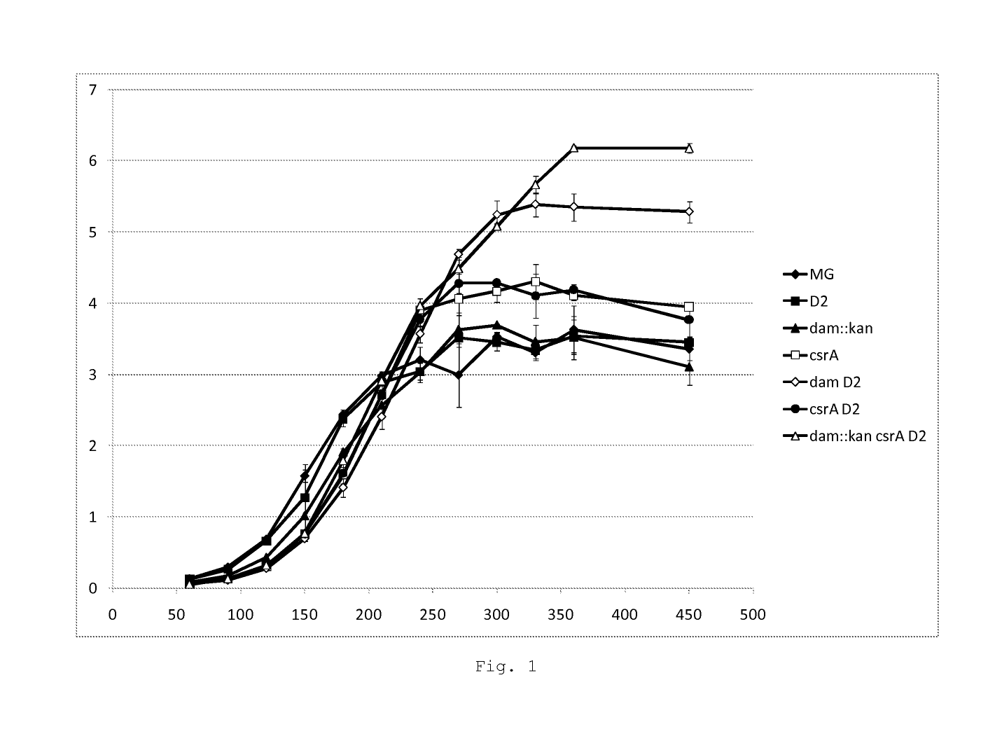

The inventors found that the hyperproliferative cell of the present invention outperforms the cell they have previously developed. Indeed, the OD600 measured was about 20% higher for the hyperproliferative cell of the present invention (see also FIG. 1).

The inventors further noticed that the combined mutant, instead of being adversely affected by the addition of mutation, have further a 10 to 20% additional OD600 values.

### Example 4

### A Method for Increasing the Plasmid Amount in a Cell

The inventors have checked whether the mutations in tldD and tldE genes and/or the mutation in Dam gene would result into a reduced copy number of plasmid per cells and the inventors searched for the plasmid content in mutated cells by comparison to the corresponding wild-type cells.

Instead of a reduced plasmid content in the (hyperproliferative) cells having TldD and/or TldE mutation combined with Dam mutation (such as the truncated Dam of the present invention) and grown in glucose (04%; w:v)-containing LB medium, the inventors measured a 3- to 20-fold increase of the plasmid amount as normalized to the cell (chromosomal) DNA (i.e. relative abundance).

The inventors further noticed a tendency towards higher values (from 20% to 100% additional increase) for low-copy number plasmids, such as plasmids with less than 50, 20, 15, 10 or even 5 copies per cell.

The inventors then compared these results with the results obtained using cells having been submitted to tldD and/or tldE mutation (such as the full deletion of tldD and/or tldE), but no mutation in Dam or in CsrA proteins. The inventors found that even this TldD and TldE mutated cell has the same phenotype of increased plasmid amount, although the increased amount was only three- to five-times the amount of the wild type cells, when the calculation was normalized with the cell DNA.

The skilled one may easily adapt the present invention to other micro-organisms and to other industrial applications.

1. A method for increasing growth of a prokaryotic cell, wherein said cell comprising tldD and/or tldE and dam sequences is submitted to a partial or total deletion of said tldD and/or tldE, and of dam nucleotide sequence(s) or to one or more mutation(s) inactivating activity of said TldD and/or TldE, and Dam proteins.

2. The method of claim 1, wherein the deletion of a 3′ part of the dam nucleotide sequence results in a dam 1-178 sequence comprising a 5′ region of the dam open reading frame starting from nucleotide 1 up to nucleotide 178 encoding a Dam 1-59 polypeptide.

3. The method according to claim 1, wherein acetate production by the cell is reduced by a factor of at least 10%.

4. The method according to claim 1 wherein the glycogen content in the cell is increased by at least 10 fold.

5. A method for increasing plasmid amount in a cell comprising the step of submitting tldD and/or tldE gene(s) to one or more mutation(s) inactivating TldD and/or TldE proteins.

6. The method of claim 5 further comprising the step of submitting a prokaryotic cell to a partial deletion or to a total deletion of dam nucleotide sequence or to one or more mutation(s) inactivating activity of a produced Dam protein.

7. A method for reducing acetate production by a cell comprising the step of submitting tldD and/or tldE gene(s) and dam gene to one or more mutation(s) inactivating TldD and/or TldE, and Dam proteins.

8. A method for increasing glycogen content in a cell comprising the step of submitting tldD and/or tldE gene(s) and dam gene to one or more mutation(s) inactivating TldD and/or TldE, and Dam proteins.

9. The method according to claim 1, wherein the cell is grown in a medium supplemented with a carbon source.

10. The method according to the claim 9, wherein the carbon source is selected from the group consisting of glycolytic carbon source and/or selected from the group consisting of glycerol, pyruvate and/or a glycolytic saccharide.

11. The method according to claim 1, further comprising the step of submitting the prokaryotic cell to a partial deletion or to a total deletion of csrA nucleotide sequence or to one or more mutation(s) inactivating activity of a produced CsrA protein.

12. The method according to claim 1, wherein the cell is a bacterial cell.

13. The method of claim 12, wherein the bacterial cell is Escherichia coli.

14. The method of claim 12, wherein the bacterial cell is Clostridium sp.

15. The method of claim 12, wherein the bacterial cell is Sphingomonas sp.

16. A recombinant cell, wherein the tldD and/or tldE, and dam nucleotide sequence(s) are partially deleted or totally deleted or comprise one or more mutation(s) inactivating the activity of produced TldD and/or TldE, and Dam proteins.

17. The recombinant cell of claim 16 further comprising an exogenous insert or an exogenous nucleotide sequence encoding a gene product of interest.

18. The recombinant cell of claim 16 the cell having been submitted to one or more mutation(s) reducing activity of CsrA protein.

19. The cell according to claim 16, comprising a prokaryote cell being not E. coli.

20. The cell of claim 19 which is selected from the group consisting of Lactobacillus sp., Bifidobacterium sp., Clostridium sp., Sphingomonas sp. and Lactococcus lactis.

21. A method of using the cell according to claim 16 comprising producing endogenous or exogenous compounds.

22. A method of using the cell according to claim 16 comprising producing glycogen.

23. A method of using the cell according to claim 16 for comprising producing plasmid.
